(19) United States
(12) Patent Application Publication
(10) Pub. No.: US 2024/0191410 A1
(43) Pub. Date: Jun. 13, 2024

(54) METHOD FOR PRODUCING OPEN-POROUS BONE IMPLANTS MADE FROM FIBERS, WITH FREELY ACCESSIBLE GUIDE STRUCTURES MADE FROM FIBERS, WHICH ARE FORMED FROM A BIOCOMPATIBLE METAL OR METAL ALLOY

(71) Applicants: Matthias RÜGER, Herrliberg (CH); Fraunhofer-Gesellschaft zur Förderung der Angewandten Forschung E.V., Munich (DE)

(72) Inventors: Olaf ANDERSEN, Dresden (DE); Cris KOSTMANN, Dresden (DE); Peter QUADBECK, Dresden (DE); Marcel FINK, Dresden (DE); Matthias RÜGER, Herrliberg (CH)

(21) Appl. No.: 18/553,998

(22) PCT Filed: Mar. 29, 2022
(86) PCT No.: PCT/EP2022/058194

§ 371 Date: Oct. 5, 2023

(30) Foreign Application Priority Data
Apr. 6, 2021 (DE) 10 2021 203 391.1

(51) Int. Cl. D04H 3/002; A61L 27/06; A61L 27/56; B22F 3/10; B22F 3/26; B32B 5/02; B32B 5/06; B32B 5/12; B32B 5/26; B32B 7/022; B32B 37/06; B32B 37/16; B32B 38/00; B32B 38/08; B32B 38/18; D04H 3/02; D04H 3/105; D04H 3/14

(52) U.S. Cl. CPC D04H 3/002; A61L 27/06; A61L 27/56; B22F 3/10; B22F 3/26; B32B 5/022; B32B 5/06; B32B 5/12; B32B 5/266; B32B 7/022; B32B 37/06; B32B 37/16; B32B 38/0012; B32B 38/08; B32B 38/1833; D04H 3/02; D04H 3/105; D04H 3/14; A61L 2430/02; B22F 2998/10; B32B 2250/20; B32B 2262/103; B32B 2305/20; B32B 2311/18; B32B 2535/00; D10B 2101/20; D10B 2401/10; D10B 2509/00

(57) ABSTRACT

In the method for producing open-porous bone implants with freely accessible guide structures made from fibers, which are formed from a biocompatible metal or metal alloy, long fibers are superimposed in multiple layers, each in the form of a nonwoven, in which the fibers in each layer are arranged in a mutually preferred axial direction. Needling is carried out in at least one of the layers, by means of which individual fibers of the respective layer are aligned in an axial direction which differs by at least 60° from the preferred axial direction in which the other fibers of the layer are aligned. The superimposed layers are materially fitted to one another point by point via sinter bridges on fibers by sintering in a heating device.

The invention relates to a method for producing open-porous bone implants made from fibers, with freely accessible guide structures made from fibers, which are formed from a biocompatible metal or metal alloy. The metal is preferably titanium or a titanium alloy, i.e., a metal, due to the use of which no physiological impairment of a patient occurs or is to be feared.

The treatment of larger bone defects is still, as previously, an insufficiently solved problem in the field of medicine. An implant must thereby provide a guide structure for bone growth, and also enable sufficient cell colonization for older patients, for whom the healing process is strongly decelerated due to previous osteoarthritic diseases, reduced cellular activity, or degenerated microarchitecture in the bones. At the same time, the implant must quickly and reliably guarantee the mechanical strength for a fast post-operative load capacity and should be biomechanically adapted to the respective load situation.

Porous implants are thus usually produced, for example, by means of selective laser melting. However, structures in the order of 20 μm are not producible using this method.

The prior art also includes the production of titanium fiber structures using sintering. In the published approaches, the sintered density is thereby varied, for example, by changing the temperature-time programs during the heat treatment for sintering. Titanium structures based on long fibers have likewise already been produced. Ungraded porous titanium fiber structures with the usual and typical mechanical properties have additionally been achieved by sintering fibers having fiber diameters of 70 μm-120 μm and porosities of 40%-80%.

The possible benefit of ungraded and disordered tissue structures for surgical use has already been demonstrated. Animal studies have shown particularly good ingrowth behavior when such bodies are used as vertebral body replacements. Increased porosity and larger pore sizes thereby have a positive effect on osteoinductivity. It has been shown in further testing that highly porous titanium fiber structures with 60%, 78% and 87% porosity result in improved ingrowth behavior. In particular, less differentiation in osteoclasts could be observed at a porosity of 87%. It is known that sintered fiber structures may have a strongly anisotropic structure with similar anisotropic properties.

It could also be demonstrated that a structural grading of biomaterial using nonmetallic, porous materials may be a success factor for bone ingrowth. Corresponding demonstrations could be carried out on graded HA scaffolds (hydroxyapatite). In the past, gradations in metallic fiber structures were realized by using different pressing processes in conjunction with variable sintering conditions. The structures produced in this way only offer a few possibilities for influencing the structure in a targeted way.

In addition, sintered porous fiber structures have only had a limited mechanical strength up to now. A consideration of certain loads in the implanted state, which, in particular, may also be direction-dependent, has been insufficient up to now.

It is therefore the object of the invention to provide bone implants which have good ingrowth behavior and improved strength, in particular also directionally dependent strength, when they are implanted and when they have been implanted.

This problem is solved according to the invention by the method which has the features of claim 1. Advantageous embodiments and refinements of the invention may be realized by features identified in the dependent claims.

In the method according to the invention, long fibers of the metal or the metal alloy are first arranged in multiple layers, each in the form of a nonwoven, in which fibers in each layer are arranged in a mutually preferred axial direction.

Chased fibers in the form of needled nonwovens with a defined, variable weight per unit area may be used as the base product for the construction of the porous structures. The production of these nonwovens may be carried out as follows:

Chasing is a mechanical production method for generating long metallic fibers. Using specially-shaped, static cutting knives, to which a starting wire with an approximate diameter of 3 mm is guided, long fibers in the range from 60 μm to 100 μm are machined, taken up, and rolled into a roll (coil).

Long fibers should have a minimum length of 20 mm, preferably 50 mm, and particularly preferably more than 70 mm.

This nonwoven with a width of approximately 100 mm may then be subsequently unrolled again. The starting wire may be deflected multiple times and guided to the knives up to a width of 100 mm. Layers obtained from a nonwoven may be cut into pieces with respectively defined lengths. These sections are superimposed and subsequently needled on a machine.

The term “needling” also includes methods for vertical cross-linking which function based on water jets. Such a method might likewise be used for vertical cross-linking of fibers. Using the kinetic energy of one or more water jets directed locally at a nonwoven fabric, the axial alignment of individual fibers in this area may be changed. By this means, the placement of individual fibers may be modified and thus deviate from the preferred axial direction of the respective layer. Needled fibers may preferably be aligned perpendicular to the preferred axial direction of the fibers of the respective layer such that these fibers are aligned from one surface across the entire thickness of a layer and optionally beyond. Multiple superimposed layers may also be positively connected to each other by needling.

Mechanical techniques may also be used for needling instead of water jets.

In the case of mechanical needling, needles fixed on a holder may plunge into the fiber stack and press some fibers down into the fiber stack with a preferred axial direction. There is a barb at the lower end of the needles, which pulls the fibers in the opposite direction when the respective needle is withdrawn.

Entanglements may form due to the relative movements of the needles, such that a bonded, multilayer nonwoven is produced in the form of a layer with a predetermined mass per unit area. The mass per unit area of an implant may be adjusted depending on the number of layers.

The starting structure for producing the implant may be obtained by stacking multiple needled nonwovens in different alignments of the fibers. Each individually needled nonwoven may be formed with long fibers having an overwhelmingly parallel alignment of the fibers to one another.

In the method according to the invention, needling is carried out in at least one of the layers, by means of which the individual fibers of the respective layer are aligned in an axial direction which differs by at least 60°, preferably by 90°, from the preferred axial direction in which the other fibers of the layer are aligned. Using the fibers aligned in this way by needling, the nonwoven may be strengthened, since these fibers may be guided from one surface of a layer to at least the immediate vicinity of the opposite surface, and thereby a connection may be established across at least almost the entire thickness of the respective layer.

The superimposed layers are then materially fitted to one another point by point via sinter bridges on fibers by sintering in a heating device under suitable atmospheric conditions. A point by point sintering of fibers may also be achieved in the fibers of one layer, which applies in particular to fibers that have been subjected to a change in direction due to needling, with fibers that are aligned in the preferred axial direction.

A preferred axial direction of the fibers of a layer may thereby mean that the fibers in the respective layer should be aligned at least almost parallel to one another, insofar as this is possible and realizable with appropriate effort.

Fibers should be used that have sufficient length. The minimum length should thereby be at least 70% of the length of an implant to be produced. However, longer fiber lengths are preferred. This relates, in particular, to applications in which implants are made from a semi-finished product or from layers, which have been prepared in advance in the form of a roll.

Two or more than two layers may be superimposed. Needling may be advantageously carried out in each of the layers. In the case of needling, at least 2% of the fibers in a layer should have had their alignment changed.

At least two directly superimposed layers may also be advantageously needled together, in that fibers of one layer are pressed into fibers of another layer and correspondingly aligned.

Layers, whose preferred axial direction, in which the respective fibers are aligned, differs from one another by at least 45º, may also be superimposed. By this means, the strength of an implant, formed using fibers aligned in this way, may be positively influenced.

The possibility also exists that layers with different densities in which the fibers are arranged, different porosities, and/or different thicknesses are superimposed prior to sintering. A graded structure with areas of increased strength and areas of increased porosity and lower strength may thus be obtained.

In the case of sintering, it is advantageous before and during sintering that the superimposed layers are subjected to compressive force by two opposite surfaces which are aligned perpendicular to the preferred axial directions in which the fibers of the layers are aligned. The sintering behavior may be positively influenced by this means, as fibers are pressed directly onto one another during sintering and a better point of contact are achieved at individually contacting fibers. In the simplest case, the superimposed layers may be arranged between sufficiently strong and temperature-stable cover layers and pads, for example, suitable sintering substrates, and a weighted pad with sufficient mass may additionally be used on this stack for support during sintering.

A constant total thickness of the superimposed layers is to be maintained during sintering, such that a predetermined thickness may be maintained for the implants, for which reason spacers may be used, which may be arranged between the sintered substrates.

A semi-finished product may be produced using the layers that are superimposed and sintered together. From the semi-finished product, at least one bone implant may then be separated from the respective semi-finished product using a separating process and brought into shape.

The separation may be carried out by conventional mechanical processing methods. Prior to the separation, the interior of the semi-finished product may thereby be filled with an infiltrate, and the separation may be carried out after the hardening of the infiltrate, and the infiltrate may be removed again after the separation is carried out. A non-crosslinking polymer or hard wax, for example, may be used as the infiltrate. The infiltrates may be removed chemically using an organic solvent, or by using a thermal treatment that may be carried out in an air atmosphere. A maximum temperature of 390° C. should not be exceeded in the case of the thermal treatment.

Biomechanically load-optimized structures with directionally defined guide structures for the ingrowth of bone cells may be provided using the implant material produced according to the invention. In materials engineering, titanium, which combines a very high mechanical strength and good fatigue properties with high biocompatibility, is the gold standard for implants.

The realization of defined anisotropic properties in a horizontal plane may be achieved, in particular, by using long titanium fibers, due to defined preferred directions of the fiber alignments.

In the vertical direction, graded pore structures may be obtained by the stratified laying of layers with different preferred axial directions of the respective fibers. The unsolved problem of reduced shear strength, caused on the one hand by the layered production and partially caused on the other hand by low density, may be solved by the invention through targeted, almost vertical needling and subsequent sintering of the anisotropic and graded volume bodies.

A complex mechanical response to external stress situations may be achieved by stratified layering of fibers with different fiber thicknesses and different degrees of vertical cross-linking. Very thin fibers with diameters of <20 μm may thereby be processed and graded pore structures with correspondingly small structure sizes may thus be realized, which are not producible in this form using other methods.

Using this type of combination of horizontal and vertical structuring, directed guide structures with very fine pore structures may be designed, which, due to directed capillary forces lead to an improved inner wetting by bodily fluids and thus facilitates the colonization with cells. The alignment of the fibers in the individual layers leads to defined anisotropic mechanical properties, such that the mechanical response of the finished implant to external stress forces may lead to preferred directions of the resulting micro-deformations. Due to the mechanoperceptive properties of bone cells, these elastic and microplastic deformations lead to a targeted formation of new bone and neovascularization. By this means, the complex, graded microstructure, which is adjustable in the horizontal and vertical direction in a targeted way, allows a targeted, adjustable growth direction of bone cells in the implant material.

The advantage of a novel approach to producing implants, which may be optimized both structurally and also mechanically and biologically, especially in the presence of decelerated healing processes, arises from the adjustable anisotropic mechanical properties based on layered, needled, and sintered metal fiber strands. The stress-shielding effect may be individually counteracted by the adjustable rigidity between biomaterials and the surrounding bone at the implantation site, in particular in areas with high mechanical stress.

1.-12. (canceled)

13. A method for producing open-porous bone implants, with freely accessible guide structures made from fibers, which are formed from a biocompatible metal or metal alloy, in which
long fibers are superimposed in multiple layers, each in the form of a nonwoven, in which the fibers in each layer are arranged in a mutually preferred axial direction, and
needling is carried out in at least one of the layers, by means of which individual fibers of the respective layer are aligned in an axial direction which differs by at least 60° from the preferred axial direction in which the other fibers of the layer are aligned, and
the superimposed layers are materially fitted to one another point by point via sinter bridges on fibers by sintering in a heating device.

14. The method according to claim 13, characterized in that layers, whose preferred axial direction, in which the respective fibers are aligned, differs from one another by at least 45°, are superimposed.

15. The method according to claim 13, characterized in that fibers of different layers, which are directly superimposed, are needled together.

16. The method according to claim 13, characterized in that layers with different densities and/or thickness in which the fibers are arranged and different porosities are superimposed prior to sintering.

17. The method according to claim 13, characterized in that, before and during sintering, the superimposed layers are subjected to compressive force by two opposite surfaces which are aligned perpendicular to the preferred axial directions in which the fibers of the layers are aligned.

18. The method according to claim 13, characterized in that a constant total thickness of the superimposed layers is maintained during sintering.

19. The method according to claim 13, characterized in that spacers are used to maintain the constant total thickness.

20. The method according to claim 13, characterized in that a semi-finished product is produced using the layers that are superimposed and sintered together, and at least one bone implant is separated from the respective semi-finished product using a separating process and brought into shape.

21. The method according to claim 13, characterized in that, prior to the separation, the interior of the semi-finished product is filled with an infiltrate, and the separation is carried out after the hardening of the infiltrate, and the infiltrate is removed again after the separation is carried out.

22. The method according to claim 13, characterized in that a non-crosslinking polymer, which is removed with a solvent, is used as the infiltrate.

23. The method according to claim 21, characterized in that a hard wax, which is thermally liquefied again and removed, is used as the infiltrate.

24. The method according to claim 21, characterized in that residual infiltrates are removed by thermal evacuation at a maximum temperature of 390° C. in an atmosphere containing air.
